(19) United States
(12) Patent Application Publication
(10) Pub. No.: US 2010/0116268 A1
(43) Pub. Date: May 13, 2010

(54) AQUEOUS AEROSOL PREPARATIONS CONTAINING THERAPEUTICALLY ACTIVE MICRO-ORGANISMS OR PARTS OF MICRO-ORGANISMS AND METHOD FOR PRODUCING CORRESPONDING AEROSOLS

(75) Inventors: Georg Boeck, Laupheim (DE); Michael Spallek, Ingelheim am Rhein (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim am Rhein (DE)

(21) Appl. No.: 12/598,145

(22) PCT Filed: Apr. 29, 2008
(86) PCT No.: PCT/EP08/55251

§ 371 Date: Dec. 28, 2009

(30) Foreign Application Priority Data
May 2, 2007 (DE) 102007020578.5

(51) Int. Cl. A61K 35/74; A61P 11/00; A61M 11/00

(52) U.S. Cl. 128/200.14; 424/93.4; 424/93.46; 424/93.42; 424/93.47

(57) ABSTRACT

The invention relates to aqueous aerosol preparations for inhalation, containing therapeutically active micro-organisms or parts of micro-organisms as the active ingredient.

The invention relates to aqueous aerosol preparations for inhalative use containing therapeutically effective micro-organisms or parts of micro-organisms as active substance.

The use of medicaments in the form of inhalable aerosols has long been known. Such aerosols are used not only for the treatment of respiratory complaints such as asthma; they are also used when the lungs or nasal mucosa are to be the organ of absorption. Frequently, the blood levels obtained with the active substances are high enough to treat diseases in other parts of the body. Inhalable aerosols may also be used as vaccines.

A number of methods are used in practice to prepare aerosols. Either suspensions or solutions of active substances are sprayed, using propellant gases inter alia, or active substances in the form of micronised powders are subjected to a vortex in the air breathed in or finally aqueous solutions are atomised using atomisers.

In molecules of a more complex structure, such as interferons, for example, the atomisation of aqueous solutions may easily lead to an undesirable reduction in the activity of the active substance, presumably as a result of shear forces and heating. It is suspected that the formation of less active protein aggregates plays a part in this process. In their article “Stability of recombinant consensus interferon to air-jet and ultrasonic atomisation”, J. Pharm. Sci. 84:1210-1214 [1995], A. Y. Ip and colleagues described examples of the formation of interferon aggregates after ultrasound or nozzle spraying with a concomitant loss of the biological activity of the interferon. Even if the destruction of the biomolecule is not complete, the reduction in activity described here is important as it causes greater consumption of the possibly expensive biomolecule and allows the dosage of active medicament per spray to become inaccurate. This reduction in the activity of molecules of a complicated structure during the aerosol production is not limited to interferons but also occurs to a greater or lesser extent during the aerosol spraying of other proteins (cf. e.g. Niven et al, Pharm Res. 12: 53-59 [1995]) and biomolecules, particularly macromolecules of this kind.

It is also known to treat mucoviscidosis patients with sulphide bridge cleaving enzymes by atomising these enzymes.

EP 1003478 describes aqueous aerosol preparations with biologically active macromolecules for propellant-free production of inhalable aerosols.

Besides the industrial production of the aerosol containing the biomolecule, a second step is needed to ensure that the biomolecules are absorbed in the lungs. The lung in an adult human has a large absorption surface but also has a number of obstacles to the pulmonary absorption of biomolecules. After inspiration through the nose or mouth, air (with the aerosol that it carries) passes into the trachea and then through progressively smaller bronchi and bronchioles into the alveoli. The alveoli have a much large surface area than the trachea, bronchi and bronchioles put together. They are the main absorption zone, not only for oxygen but also for biologically active macromolecules. In order to pass from the air into the bloodstream, molecules have to cross the alveolar epithelium, the capillary endothelium and the lymph-containing interstitial space between these two layers of cells. This may take place by active or passive transport processes. The cells in these two layers of cells are close together, so that most of the large biological macromolecules (such as proteins, for example) cross this barrier much more slowly than smaller molecules. The process of crossing the alveolar epithelium and the capillary endothelium competes with other biological processes that lead to the destruction of the biomolecule. The bronchoalveolar liquid contains exoproteases [cf. e.g. Wall D. A. and Lanutti, A. T. ‘High levels of exopeptidase activity are present in rat and canine bronchoalveolar lavage fluid’. Int. J. Pharm. 97:171-181 (1993)]. It also contains macrophages which eliminate inhaled protein particles by phagocytosis. These macrophages migrate to the base of the bronchial tree, from where they are expelled from the lungs by the mucociliary clearance mechanism. They may then migrate into the lymphatic system. Moreover, the macrophages may be influenced by the aerosolised protein in their physiology, e.g. interferons may activate alveolar macrophages. The migration of activated macrophages is a further mechanism for propagating the systemic effect of an inhaled protein. The complexity of this process means that results of aerosol tests with one type of protein can only be transferred to another type of protein to a limited extent. Small differences between interferons may for example have a significant influence on their susceptibility to the degradation mechanisms in the lungs [cf. Bocci et al ‘Pulmonary catabolism of interferons: alveolar absorption of ¹²⁵-I labelled human interferon alpha is accompanied by partial loss of biological activity’ Antiviral Research 4:211-220 (1984)].

Micro-organisms, which are a form of biological macromolecules, may indeed be atomised in principle, but these atomisation generally takes place with a loss of activity. The definition of micro-organisms in this context encompasses all tiny single-cell living organisms with a size of ≦200 μm. Micro-organisms include in particular bacteria and fungi.

The aim of the present invention is to provide aqueous aerosol preparations which contain therapeutically effective micro-organisms or parts of micro-organisms, particularly bacteria, fungi or parts thereof, as active substance and can be used for inhalation.

Surprisingly it has been found that liquid preparations of therapeutically active micro-organisms or parts of micro-organisms as active substance can be atomised without any appreciable loss of activity.

Preferably the atomisers used are propellant-free atomisers which spray a predetermined amount of an aerosol preparation at high pressure between 100 and 500 bar through at least one nozzle with a hydraulic diameter of 1 to 12 microns, so as to obtain inhalable droplets with an average particle size of less than 10 microns.

Moreover, highly concentrated solutions of therapeutically effective micro-organisms or parts of micro-organisms may also be atomised. The use of highly concentrated solutions makes it possible to use a device with a number of single doses in a small reservoir.

The invention also relates to aerosol preparations in the form of aqueous solutions which contain, as active substance, therapeutically effective micro-organisms or parts of micro-organisms, particularly therapeutically effective bacteria, fungi or parts of bacteria or fungi, in an amount of between 3 mg/ml and 100 mg/ml. Particularly preferred are the micro-organisms of the Bacillus, Staphylococcus, Pseudomonas, Escherichia, Salmonella, Candida or Aspergillus strains or a mixture of these strains. Most particularly preferred are micro-organisms of the types Bacillus subtilis, Staphylococcus aureus, Pseudomonas aeruginosa, Escherichia coli, Salmonella abony, Candida albicans, Aspergillus niger, or a mixture of these types.

Surprisingly it has been found that more viscous solutions of therapeutically active micro-organisms or parts of micro-organisms may be sprayed to form inhalable droplets of a suitable droplet size.

This allows larger amounts of active substance to be administered in each dose and thus increases the therapeutic efficacy of therapeutically active micro-organisms or parts of micro-organisms in inhalative therapy.

For the aerosol according to the invention, therapeutically effective micro-organisms or parts of micro-organisms containing aqueous aerosol preparations up to a limiting viscosity of 1600×10⁻⁶ Pascal are used.

More highly viscous solutions of therapeutically active micro-organisms or parts of micro-organisms, having a limiting viscosity of up to 1100×10⁻⁶ Pascal, are preferred. The limiting viscosities stated were determined using an Oswald viscosimeter by a method known from the literature. As a comparison: the limiting viscosity of water is 900×10⁻⁶ Pascal.

The aqueous aerosol preparation may also contain one or more adjuvants selected from among the surfactants, emulsifiers, stabilisers, permeation promoters and/or preservatives as well as an amino acid to improve the solubility/stability of the active substance, preferably proline.

The invention also relates to the use of the aqueous aerosol preparation for the treatment of respiratory complaints, particularly for the treatment of chronic obstructive pulmonary disease (COPD) or for the immune treatment of humans and animals.

The atomiser used may be any of the conventional devices, with or without propellant gas.

A new generation of propellant-free atomisers is described in U.S. Pat. No. 5,497,944 and WO 97/12687, the contents of which are hereby incorporated by reference. A preferred nozzle arrangement for nebulising the aqueous aerosol preparations of biologically active macromolecules according to the invention is shown in FIG. 8 of the U.S. patent. The particular advantage of the nebulisers described therein is that no propellant gases are used.

A further developed embodiment of the atomisers described therein is disclosed in PCT/EP96/04351=WO 97/12687. In relation to the present invention reference is made expressly to FIG. 6 described therein and the associated parts of the description of the application. The nebuliser described therein may advantageously be used to produce the claimed inhalable aerosols of biologically active macromolecules. In the nebulisers described therein, active substance-containing solutions of defined volumes are sprayed through small nozzles at high pressures, so as to obtain inhalable aerosols with an average particle size of between 3 and 10 microns.

Of particular importance is the use of the device described in the above-mentioned patent or patent application for propellant-free atomisation of the aerosol preparation according to the invention. The atomiser (nebuliser) essentially consists of the upper housing part, a pump housing, a nozzle, a locking clamp, a spring housing, a spring and a storage container, characterised by
a pump housing fixed in the upper housing part and carrying at one end a nozzle body with the nozzle,
a hollow piston with valve body,
a power take-off flange in which the hollow body is fixed and which is located in the upper housing part,
a locking clamping mechanism located in the upper housing part,
a spring housing with the spring located therein, which is rotatably mounted on the upper housing part by means of a rotary bearing,
a lower housing part which is fitted onto the spring housing in the axial direction.

The hollow piston with valve body corresponds one of the above-mentioned devices. It projects partially into the cylinder of the pump housing and is disposed to be axially movable in the cylinder. At the moment of release of the spring the hollow piston with valve body exerts, at its high pressure end, a pressure of 5 to 60 Mpa (about 50 to 600 bar), preferably 10 to 60 Mpa (about 100 to 600 bar) on the fluid.

The nozzle in the nozzle body is preferably microstructured, i.e. manufactured by micro-engineering. Microstructured nozzle bodies are disclosed for example in WO-94/07607; reference is hereby made to the contents of this specification.

The nozzle body consists for example of two sheets of glass and/or silicon securely fixed together, at least one of which has one or more microstructured channels which connect the nozzle inlet end to the nozzle outlet end. At the nozzle outlet end there is at least one round or non-round opening less than or equal to 10 μm.

The directions of spraying of the nozzles in the nozzle body may run parallel to each other or may be inclined relative to one another. In the case of a nozzle body having at least two nozzle openings at the outlet end, the directions of spraying may be inclined relative to one another at an angle of 20 degrees to 160 degrees, preferably at an angle of 60 to 150 degrees.

The directions of spraying meet in the region of the nozzle openings.

The valve body is preferably mounted at the end of the hollow piston which faces the nozzle body.

The locking clamping mechanism contains a spring, preferably a cylindrical helical compression spring as a store for the mechanical energy. The spring acts on the power take-off flange as a spring member the movement of which is determined by the position of a locking member. The travel of the power take-off flange is precisely limited by an upper stop and a lower stop. The spring is preferably tensioned via a stepping-up gear, e.g. a helical sliding gear, by an external torque which is generated when the upper housing part is turned relative to the spring housing in the lower housing part. In this case, the upper housing part and the power take-off flange contain a single- or multi-speed spline gear.

The locking member with engaging locking surfaces is arranged in an annular configuration around the power take-off flange. It consists for example of a ring of plastics or metal which is inherently radially elastically deformable. The ring is arranged in a plane perpendicular to the axis of the atomiser. After the locking of the spring, the locking surfaces of the locking member slide into the path of the power take-off flange and prevent the spring from being released. The locking member is actuated by means of a button. The actuating button is connected or coupled to the locking member. In order to actuate the locking clamping mechanism the actuating button is moved parallel to the annular plane, preferably into the atomiser, and the deformable ring is thereby deformed in the annular plane.

The lower housing part is pushed axially over the spring housing and covers the bearing, the drive for the spindle and the storage container for the fluid.

When the atomiser is operated, the upper part of the housing is rotated relative to the lower part, the lower part taking the spring housing with it. The spring meanwhile is compressed and biased by means of the helical sliding gear, and the clamping mechanism engages automatically. The angle of rotation is preferably a whole-number fraction of 360 degrees, e.g. 180 degrees. At the same time as the spring is tensioned, the power take-off component in the upper housing part is moved along by a given amount, the hollow piston is pulled back inside the cylinder in the pump housing, as a result of which some of the fluid from the storage container is sucked into the high pressure chamber in front of the nozzle.

If desired, a plurality of replaceable storage containers containing the fluid to be atomised can be inserted in the atomiser one after another and then used. The storage container contains the aqueous aerosol preparation according to the invention.

The effectiveness of a nebulisation device can be tested in an in vitro system, by nebulising a protein solution and catching and analysing the aerosol. The activity of the protein in the nebulisation solution (a) is compared with the activity in the analysed aerosol (b), e.g. by means of an immunoassay or an assay of the biological activity of the protein. This experiment makes it possible to evaluate the degree of destruction of the protein by the nebulisation process.

A second parameter for evaluating the aerosol quality is the so-called inhalable fraction which is defined here as the proportion of droplets of mist with a mass median aerodynamic diameter (MMAD) of less than 5.8 μm. The MMAD may be measured e.g. using an “Andersen Cascade Impactor”. For efficient protein absorption it is important not only to achieve nebulisation with no appreciable loss of activity but also to generate an aerosol with a good (approx. 60%) inhalable fraction. Aerosols with an MMAD of less than 5.8 μm are significantly more suitable for reaching the alveoli, their chances of being absorbed being clearly greater on account of the very great absorbent surface. The effectiveness of a nebulising device can also be tested in an in vivo system, in which case factors such as susceptibility to lung proteases come into play. As an example of an in vivo test system, a protein-containing mist may be administered to a dog through a tracheal tube. Blood samples are taken at suitable intervals and then the protein level in the plasma is measured using immunological or biological methods.

The following in vivo tests are described to illustrate advantages of the aerosol according to the invention.

In Vitro Tests with the Soft Inhaler Respimat®

The reservoir of a Respimat device (a) was filled in each case with a suspension of different micro-organisms in 50 mM trisodium citrate, 150 mM NaCl, pH 5.5. The following micro-organisms were used:
1.) Bacillus subtilis ATCC 6633
2.) Staphylococcus aureus ATCC 6538
3.) Pseudomonas aeruginosa ATCC 9027
4.) Escherichia coli ATCC 8739
5.) Salmonella abony NCTC 6017
6.) Candida albicans ATCC 10231
7.) Aspergillus niger ATCC 16404
   These strains are deposited with the American Tissue Culture Collection.

A number of sprays corresponding to a total volume of approx. 0.5 ml of were released using the Respimat®. The aerosol produced was captured in a sealed 1000 ml shaking flask with 100 ml of a physiological buffer solution and 0.1% Tween 80. Then the flask was sealed off completely at its opening and the aerosol was taken up in the buffer solution in the flask by gentle shaking.

The amount of aerosol released was determined by weighing the Respimat® inhaler. The subsequent microbiological tests were carried out according to the instructions in Ph. Eur. 3, 2000 (2.6.12) and USP 24:

20 ml of the buffer solution from the shaking flask and one aliquot of 0.1 ml of the [noun omitted] in the reservoir of the Respimat® inhaler were filtered through membrane filters separately from one another. As a control batch, 0.1 ml of the corresponding suspension of the above-mentioned micro-organisms in 20 ml buffer solution are filtered.

The membrane filters through which the suspensions of bacteria were filtered were placed on agar plates after the filtration and incubated for 5 days at 33° C.

The membrane filters through which the suspensions of yeasts and fungi were filtered were placed on agar plates after the filtration and incubated for 5 days at 25° C. In all, three tests are carried out on each micro-organism. Tab. 1, Tab. 3 and Tab. 5 show the results of the three tests. As a comparison, the colony-forming units per millilitre (CFU/ml) from the aerosol, the reservoir and the control group are shown. The survival rate and death rate in percent were calculated for the captured aerosol in relation to the reservoir suspension.

Tab. 2, Tab. 4 and Tab. 6 show the results for the amount of aerosol released, determined by weighing.

TABLE 1
Results of the first test.

| | | | control | survival | death |
|---|---|---|---|---|---|
| | aerosol | reservoir | group | rate | rate |
| | [CFU/ml] | [CFU/ml] | [CFU/ml] | [%] | [%] |
| Staphylococcus | 86 | 88 | 92 | 97.7 | 2.3 |
| aureus | | | | | |
| Bacillus subtilis | 60 | 62 | 64 | 96.8 | 3.2 |
| Pseudomonas | 78 | 84 | 90 | 92.9 | 7.1 |
| aeruginosa | | | | | |
| Escherichia coli | 80 | 82 | 86 | 97.6 | 2.4 |
| Salmonella abony | 64 | 68 | 70 | 94.1 | 5.9 |
| Candida albicans | 6 | 70 | 74 | 8.6 | 91.4 |
| Aspergillus niger | 0 | 42 | 50 | 0.0 | 100.0 |

TABLE 2
Measurement of the amount of aerosol released for the first test

| | Amount of aerosol |
|---|---|
| | [g] |
| Staphylococcus aureus | 0.52 |
| Bacillus subtilis | 0.54 |
| Pseudomonas aeruginosa | 0.50 |
| Escherichia coli | 0.49 |
| Salmonella abony | 0.51 |
| Candida albicans | 0.50 |
| Aspergillus niger | 0.53 |

TABLE 3
Results of the second test

| | | | control | survival | death |
|---|---|---|---|---|---|
| | aerosol | reservoir | group | rate | rate |
| | [CFU/ml] | [CFU/ml] | [CFU/ml] | [%] | [%] |
| Staphylococcus | 78 | 84 | 88 | 92.9 | 7.1 |
| aureus | | | | | |
| Bacillus subtilis | 64 | 70 | 72 | 91.4 | 8.6 |
| Pseudomonas | 76 | 84 | 82 | 90.5 | 9.5 |
| aeruginosa | | | | | |
| Escherichia coli | 70 | 74 | 80 | 94.6 | 5.4 |
| Salmonella abony | 64 | 77 | 74 | 83.1 | 16.9 |
| Candida albicans | 0 | 62 | 68 | 0.0 | 100.0 |
| Aspergillus niger | 0 | 42 | 51 | 0.0 | 100.0 |

TABLE 4
Measurement of the amount of aerosol released for the second test

| | amount of aerosol |
|---|---|
| | [g] |
| Staphylococcus aureus | 0.55 |
| Bacillus subtilis | 0.50 |
| Pseudomonas aeruginosa | 0.46 |
| Escherichia coli | 0.52 |
| Salmonella abony | 0.50 |
| Candida albicans | 0.48 |
| Aspergillus niger | 0.55 |

TABLE 5
Results of the third test

| | | | control | survival | death |
|---|---|---|---|---|---|
| | aerosol | reservoir | group | rate | rate |
| | [CFU/ml] | [CFU/ml] | [CFU/ml] | [%] | [%] |
| Staphylococcus | 74 | 78 | 84 | 94.9 | 5.1 |
| aureus | | | | | |
| Bacillus subtilis | 58 | 66 | 70 | 87.9 | 12.1 |
| Pseudomonas | 68 | 74 | 80 | 91.9 | 8.1 |
| aeruginosa | | | | | |
| Escherichia coli | 80 | 86 | 84 | 93.0 | 7.0 |
| Salmonella abony | 57 | 68 | 72 | 83.8 | 16.2 |
| Candida albicans | 0 | 64 | 71 | 0.0 | 100.0 |
| Aspergillus niger | 0 | 38 | 48 | 0.0 | 100.0 |

TABLE 6
Measurement of the amount of aerosol released for the second test

| | amount of aerosol [g] |
|---|---|
| | [g] |
| Staphylococcus aureus | 0.51 |
| Bacillus subtilis | 0.54 |
| Pseudomonas aeruginosa | 0.53 |
| Escherichia coli | 0.50 |
| Salmonella abony | 0.51 |
| Candida albicans | 0.53 |
| Aspergillus niger | 0.49 |

TABLE 7
Statistics of the survival rate over all three tests

| | Mean | SD | Min. | Max. | |
|---|---|---|---|---|---|
| | [%] | [%] | [%] | [%] | N |
| Staphylococcus aureus | 95.2 | 2.4 | 92.9 | 97.7 | 3 |
| Bacillus subtilis | 92.0 | 4.5 | 87.9 | 96.8 | 3 |
| Pseudomonas aeruginosa | 91.7 | 1.2 | 90.5 | 92.9 | 3 |
| Escherichia coli | 95.1 | 2.3 | 93.0 | 97.6 | 3 |
| Salmonella abony | 87.0 | 6.2 | 83.1 | 94.1 | 3 |
| Candida albicans | 2.9 | 4.9 | 0.0 | 8.6 | 3 |
| Aspergillus niger | 0.0 | 0.0 | 0.0 | 0.0 | 3 |

TABLE 8
Statistics of the death rate over all three tests

| | Mean | SD | Min. | Max. | |
|---|---|---|---|---|---|
| | [%] | [%] | [%] | [%] | N |
| Staphylococcus aureus | 4.8 | 2.4 | 2.3 | 7.1 | 3 |
| Bacillus subtilis | 8.0 | 4.5 | 3.2 | 12.1 | 3 |
| Pseudomonas aeruginosa | 8.3 | 1.2 | 7.1 | 9.5 | 3 |
| Escherichia coli | 4.9 | 2.3 | 2.4 | 7.0 | 3 |
| Salmonella abony | 13.0 | 6.2 | 5.9 | 16.9 | 3 |
| Candida albicans | 97.1 | 4.9 | 91.4 | 100.0 | 3 |
| Aspergillus niger | 100.0 | 0.0 | 100.0 | 100.0 | 3 |

Tab. 7 shows the statistical data of the survival rate. More than 87% surviving micro-organisms were found for Bacillus subtilis, Staphylococcus aureus, Pseudomonas aeruginosa, Escherichia coli and Salmonella abony. This means that between 87 and 95 percent of the micro-organisms that were nebulised were still capable of dividing and growing after the nebulisation and working up of the aerosol. This is an indication that the micro-organisms have survived the nebulisation.

The rates of Candida albicans and Aspergillus niger are below 3%. Tab. 8 shows the corresponding death rate. This means that between 97 and 100 percent of the micro-organisms that were nebulised were no longer capable of dividing and growing after the nebulisation and working up of the aerosol. This is an indication that the micro-organisms have either not survived the nebulisation or because of their size have been retained by the filter mechanisms in the Respimat® inhaler.

The results of the tests described above show that generally after being used and nebulised in the Respimat® inhaler bacteria show no loss of activity.

Very large micro-organisms such as e.g. yeasts and fungi (Candida albicans, Aspergillus niger) are obviously retained in the Respimat because of their size. They cannot pass through the filters of the Respimat® inhaler.

On account of the high variability in biological tests, it can be assumed that when nebulised in the Respimat® inhaler bacteria are not killed off in practice or held back by filtration. The conversion of bacterial suspensions into aerosols in the Respimat® inhaler has no effect on the vitality of the micro-organisms. Thus, bacteria or components of bacteria can be efficiently transported into the human lung for curative purposes.

1. An aqueous aerosol preparation for inhalation comprising micro-organisms or parts of micro-organisms as active substance, characterized in that the aqueous aerosol preparation comprises the active substance in a therapeutically effective form.

2. The aqueous aerosol preparation according to claim 1, characterized in that the aqueous aerosol preparation comprises the active substance in a concentration of between 3 mg/ml and 100 mg/ml.

3. The aqueous aerosol preparation according to claim 1, characterized in that micro-organisms of the genus Bacillus, Staphylococcus, Pseudomonas, Escherichia, Salmonella, or a mixture of these genera are the active substance.

4. The aqueous aerosol preparation according to claim 1, characterized in that micro-organisms of the species Bacillus subtilis, Staphylococcus aureus, Pseudomonas aeruginosa, Escherichia coli, Salmonella abony or a mixture of these species are the active substance.

5. The aqueous aerosol preparation according to claim 1, characterized in that the preparation comprises one or more adjuvants selected from among the surfactants, emulsifiers, stabilisers, permeation enhancers, and preservatives, and combinations thereof.

6. The aqueous aerosol preparation according to claim 1, characterized in that the preparation also comprises an amino acid for improving the solubility/stability of the active substance.

7. The aqueous aerosol preparation according to claim 6, characterized in that the preparation is suitable for use in a propellant-free nebulizer.

8. The aqueous aerosol preparation according to claim 7, characterized in that the preparation has a limiting viscosity of up to 1600×10⁻⁶ Pascal.

9-15. (canceled)

16. A propellant-free nebulizer with an aqueous aerosol preparation for inhalation which aqueous aerosol preparation comprises micro-organisms or parts of micro-organisms as active substance, characterized in that a single dose of the aqueous aerosol preparation is measured in a measuring chamber and is sprayed at high pressure of between 100 and 500 bar through at least one nozzle with a hydraulic diameter of 1 to 12 microns to form inhalable droplets with a particle size of less than 10 microns within a time of between one and two seconds.

17. The propellant-free nebulizer of claim 16 characterized in that the single dose is between 10 and 20 microliters.

18. The propellant-free nebulizer of claim 16 characterized in that the nebulizer has two nozzles that are directed so that the two jets meet in such a way that the aqueous aerosol preparation is nebulized.
